(19) United States
(12) Patent Application Publication
(10) Pub. No.: US 2004/0047821 A1
(43) Pub. Date: Mar. 11, 2004

(54) Cosmetic composition comprising an anionic latex, an ether having two fatty chains and a mixture of fatty alcohols

(76) Inventors: Mireille Maubru, Chatou (FR); Bernard Beauquey, Clichy (FR)

Correspondence Address: NIXON & VANDERHYE, PC, 1100 N GLEBE ROAD, 8TH FLOOR, ARLINGTON, VA, 22201-4714, US

(21) Appl. No.: 10/398,893

(22) PCT Filed: Oct. 8, 2001
(86) PCT No.: PCT/FR01/03090

(30) Foreign Application Priority Data
Oct. 13, 2000 (FR) 00/13172

(51) Int. Cl. A61K 7/021

(52) U.S. Cl. 424/63

(57) ABSTRACT

The invention concerns a cosmetic composition comprising in a cosmetically acceptable medium, at least an anionic latex, at least an ether with two fatty chains and a mixture of long-chain linear saturated fatty alcohols containing at least 50 wt. % of an alcohol which contains 22 carbon atoms. The invention further concerns a method for cosmetic treatment of keratinous materials and a use of the inventive composition a shampoo.

The present invention relates to a cosmetic composition comprising, in a cosmetically acceptable medium, at least one surfactant, at least one anionic latex, at least one ether having two fatty chains and a mixture of linear and saturated fatty alcohols, to a method for the cosmetic treatment of keratinous materials and to the use of said cosmetic composition as a shampoo.

Known combinations, in pearlescent shampoos, of anionic latexes and of glycol distearate lead to cosmetic properties which are not entirely satisfactory, in particular on wet hair.

The applicant has found, surprisingly, that the particular use of an ether having two fatty chains, which is solid at room temperature, in combination with a mixture of long chain linear and saturated fatty alcohols containing at least 50% by weight of an alcohol which contains 22 carbon atoms, and of anionic latexes, in a cosmetically acceptable medium, substantially improved cosmetic properties on wet hair, and in particular lightness.

The subject of the present invention is therefore a cosmetic composition comprising, in a cosmetically acceptable medium, at least one surfactant, at least one anionic latex, at least one ether having two fatty chains and a mixture of long chain linear and saturated fatty alcohols containing at least 50% by weight of an alcohol which contains 22 carbon atoms.

The expression cosmetically acceptable medium is understood to mean a medium compatible with all keratinous materials such as the skin, the hair, the nails, the eyelashes and the eyebrows, the lips and any other area of the body and of the face, but also having a pleasant odor, appearance and feel.

Another subject of the invention consists of a method for the cosmetic treatment of keratinous materials using the abovementioned composition.

The subject of the invention is also the use of the composition according to the invention as a shampoo.

Other subjects, characteristics, features and advantages of the invention will emerge more clearly on reading the description and the various examples which follow.

According to the invention, the cosmetic composition comprises, in a cosmetically acceptable medium, at least one surfactant, at least one anionic latex, at least one ether having two fatty chains and a mixture of long chain linear and saturated fatty alcohols containing at least 50% by weight of an alcohol which contains 22 carbon atoms.

The ethers having two fatty chains which are appropriate in the present invention are chosen in particular from ethers having two fatty chains, which are solid at a temperature of less than or equal to about 30° C., and which correspond to the following formula:

R—O—R′ (I)

in which:

R and R′, which are identical or different, denote a linear or branched, saturated or unsaturated hydrocarbon group containing from 12 to 30 carbon atoms, and preferably from 14 to 24 carbon atoms, R and R′ being chosen such that the compound of formula (I) is solid at a temperature of less than or equal to about 30° C. Preferably, R and R′ are identical and denote an alkyl group such as a stearyl group.

The ethers having two fatty chains which can be used according to the invention are insoluble in the cosmetic compositions, and may be prepared according to the method described in Patent Application DE 41 27 230.

There is used in particular, in the context of the present invention, the distearyl ether sold under the name CUTINA® STE by the company COGNIS.

According to the invention, the ether having two fatty chains may represent from 0.5% to 15% by weight, preferably from 0.5% to 5% by weight, and still more preferably from 1% to 3% by weight, relative to the total weight of the final composition.

The mixture of long chain linear and saturated fatty alcohols containing at least 50% by weight of an alcohol which contains 22 carbon atoms contains more particularly at least 70% by weight of a C₂₂ alcohol relative to the total weight of the mixture.

In general, the mixture of long chain linear and saturated fatty alcohols contains C₁₆ to C₂₄ fatty alcohols. The C₁₆ and C₂₄ fatty alcohols each generally represent less than 2% by weight, and the C₁₈ alcohols less than 10% by weight of the total weight of the mixture.

By way of examples of such fatty alcohols, there may be mentioned in particular the product marketed under the name NAFOL® 1822 C by the company CONDEA which contains about 0.5% of a C₁₆ alcohol, 4-6% of a C₁₈ alcohol, 15-19% of a C₂₀ alcohol, 74-78% of a C₂₂ alcohol and about 1.5% of a C₂₄ alcohol, or the product marketed under the name NAFOL® 2298 by the company CONDEA which contains 98% by weight of a C₂₂ alcohol.

According to the invention, the mixture of long chain linear and saturated fatty alcohols containing at least 50% by weight of alcohol which contains 22 carbon atoms may represent from 0.5% to 15% by weight, preferably from 0.5% to 5% by weight, and still more preferably from 1% to 3% by weight, relative to the total weight of the cosmetic composition.

The ether having two fatty chains/C₂₂ fatty alcohols ratio is generally between 0.2 and 8, and preferably between 0.3 and 5.

The expression anionic latex is understood to mean a colloidal dispersion of particles of anionic polymer in an aqueous or organic liquid phase.

The latexes used according to the invention are those containing anionic functional groups which are provided either by polymerization or copolymerization in emulsion or in suspension of anionic monomers, by the customary methods known to a person skilled in the art, or by combination of the free radical derived from the reaction initiator with the monomer or monomers considered during initiation of the polymerization of said monomers, or else by juxtaposition of these two methods of synthesis, or alternatively by introducing end groups through a chain transfer reaction in the second method using a reaction initiator.

The method using reaction initiators is described in particular in the article by R. M. FITCH, “Preparation and characterization of charge stabilised polymer colloids” in “Polyelectrolytes and their applications”, 51-69 by D. REIDER publishing company.

The latexes used more particularly in the compositions according to the invention result from the polymerization of one or more of the following ionogenic monomers: acrylic, methacrylic, maleic, crotonic, itaconic, para-styrenesulfonic, vinyl-sulfonic, 2-methacryloyloxyethylsulfonic and 2-acrylamido-2-methylpropylsulfonic acids, alone or as a mixture with various monomers such as styrene, butadiene, ethylene, propylene, isoprene, isobutene, vinyltoluene, vinyl propionate, vinyl alcohol, acrylonitrile, chloroprene, vinyl chloride, vinylidene chloride, vinyl acetate, urethanes, vinyl ether, vinylpyrrolidone, vinylimidazole, esters of acrylic, methacrylic, vinylacetic, maleic, crotonic or itaconic acids.

The latexes obtained according to the second method above result from the use of initiators chosen from. oxidation-reduction systems, peroxides, perphosphates, percarbonates, persulfates, peroxidized organic acids such as, for example, peracetic acid, and the persulfate-bisulfite-iron mixture.

The functional monomers used in the case of a chain transfer reaction are chosen from organic thioacids such as, for example, mercaptoacetic acid.

As a particularly preferred anionic latex of the invention, there may be used, for example, an aqueous dispersion comprising an acrylic copolymer consisting of (a) about 35 to 74% by weight of an alkyl acrylate, (b) about 25 to 65% of alkyl methacrylate and (c) about 1 to 15% of one or more ethylenic carboxylic acids or their salts, having from 3 to 5 carbon atoms, alkyl groups containing from 1 to 5 carbon atoms, these percentages being expressed by weight relative to the total weight of the copolymer.

The alkyl acrylate is preferably chosen from methyl acrylate, ethyl acrylate, propyl acrylate and butyl acrylate. Ethyl acrylate is particularly preferred.

The alkyl acrylate concentration is preferably between 40 and 70% by weight, and more particularly between 50 and 60% by weight relative to the total weight of the copolymer.

The alkyl methacrylate is preferably chosen from methyl methacrylate, ethyl methacrylate, propyl methacrylate and butyl methacrylate. Methyl methacrylate is particularly preferred.

The alkyl methacrylate concentration is preferably between 30 and 50% by weight, and more particularly between 30 and 40% by weight relative to the total weight of the copolymer.

The preferred ethylenic carboxylic acids are acrylic acid, methacrylic acid, crotonic acid, itaconic acid or mixtures thereof. Acrylic acid and methacrylic acid are particularly preferred. According to the invention, it is also possible to use salts of these carboxylic acids.

The concentration of ethylenic carboxylic acids, or of their salts, is preferably between 5 and 15% by weight, and more particularly between 8 and 12% by weight relative to the total weight of the copolymer. In a particularly preferred embodiment of the invention, acrylic acid is used with methacrylic acid, each in a concentration of between 2 and 10% by weight, the total concentration of these two acids not exceeding 15% by weight of the total weight of the copolymer.

The copolymer may also contain low quantities, that is to say less than 10%, preferably less than 5% and more particularly less than 2%, of a polymerizable monomer other than those mentioned above.

Generally, the dispersion contains at least 0.5% surfactant allowing the insoluble polymer to be prepared and maintained as a dispersion. According to the invention, it is possible to use any type of surfactant, but preferably a nonionic surfactant.

The average size of the copolymer particles in the dispersion is preferably between 0.1 and 1 micrometer.

According to a particularly preferred embodiment of the implementation of the invention, a copolymer is used which comprises from 50 to 60% by weight of ethyl acrylate, from 30 to 40% by weight of methyl methacrylate, from 2 to 10% by weight of acrylic acid, from 2 to 10% by weight of methacrylic acid, the total concentration of acrylic and methacrylic acids not exceeding 15% by weight relative to the total weight of the acrylic copolymer.

Such a copolymer is for example described in Patent Application EP-A-590604.

An aqueous dispersion of the acrylic copolymer described above, comprising 25% by weight of an ethyl acrylate/methyl methacrylate/methacrylic acid/acrylic acid copolymer having a glass transition temperature of about 30° C., is sold in particular under the trade name AMERHOLD® DR-25 by the company AMERCHOL.

The anionic latexes may be used in a quantity of between 0.05 and 20% by weight of active substances, preferably between 0.15 and 5% by weight of active substances relative to the total weight of the composition.

The surfactants which are suitable in the present invention are in particular chosen from anionic, amphoteric, nonionic or cationic surfactants well known in the art and as described below, and mixtures thereof.

As anionic surfactants which can be used, in the context of the present invention, there may be mentioned especially salts, in particular alkali metal salts such as sodium salts, ammonium salts, amine salts, amino alcohol salts or salts of alkaline earth metals, for example magnesium, of the following compounds: alkyl sulfates, alkyl ether sulfates, alkyl amidoether sulfates, alkyl aryl polyether sulfates, monoglyceride sulfates; alkyl sulfonates, alkyl amide sulfonates, alkyl aryl sulfonates, α-olefin sulfonates, paraffin sulfonates; alkyl sulfosuccinates, alkyl ether sulfosuccinates, alkyl amide sulfosuccinates; alkyl sulfoacetates; acyl sarcosinates; and acyl glutamates, the alkyl and acyl groups of all these compounds comprising from 6 to 24 carbon atoms and the aryl group preferably denoting a phenyl or benzyl group. It is also possible to use the esters of a C₆-C₂₄ alkyl and polyglycoside carboxylic acids such as alkyl glucoside citrates, alkyl polyglycoside tartrates and alkyl polyglycoside sulfosuccinates; alkyl sulfosuccinamates, acyl isethionates and N-acyltaurates, the alkyl or acyl group of all these compounds comprising from 12 to 20 carbon atoms. Among the anionic surfactants which can also be used, there may also be mentioned acyl lactylates in which the acyl group comprises from 8 to 20 carbon atoms.

In addition, there may also be mentioned alkyl D-galactoside uronic acids and their salts as well as polyoxyalkylenated (C₆-C₂₄)alkyl ether carboxylic acids, polyoxyalkylenated (C₆-C₂₄)alkyl (C₆-C₂₄)aryl ether carboxylic acids, polyoxyalkylenated (C₆-C₂₄)alkyl amidoether carboxylic acids and their salts, in particular those comprising from 2 to 50 ethylene oxide groups, and mixtures thereof.

The anionic surfactants as described above may be used alone or in the form of a mixture. There are preferably used alkyl sulfates, alkyl ether sulfates and alkyl ether carboxylates, and mixtures thereof, in particular in the form of their salts of alkali or alkaline-earth metals, of ammonium, of an amine or of an amino alcohol.

The anionic surfactant preferably used in the present invention is a sodium lauryl ether sulfate.

The amphoteric surfactants which are suitable in the invention are, for example, secondary or tertiary aliphatic amine derivatives in which the aliphatic group is a linear or branched chain comprising from 8 to 22 carbon atoms and containing at least one water-solubilizing anionic group such as, for example, a carboxylate, sulfonate, sulfate, phosphate or phosphonate group; there may also be mentioned (C₈-C₂₀)-alkylbetaines, sulfobetaines, (C₈-C₂₀)alkylamido (C₆-C₈)-alkylbetaines or (C₈-C₂₀) alkylamido (C₆-C₈) alkylsulfobetaines; and mixtures thereof.

Among the amine derivatives, there may be mentioned the products marketed under the name MIRANOL®, as described in patents U.S. Pat. No. 2,528,378 and U.S. Pat. no. 2,781,354 and classified in the CTFA dictionary, 3rd edition, 1982, under the names Amphocarboxyglycinate and Amphocarboxypropionate having the respective structures (II) and (III):

R₁—CONHCH₂CH₂—N⁺(R₂) (R₃) (CH₂COO⁻) (II)

in which:

R₁ represents an alkyl group derived from an acid R₁—COOH present in hydrolyzed copra oil, a heptyl, nonyl or undecyl group,

R₂ represents a beta-hydroxyethyl group, and

R₃ represents a carboxymethyl group; and

R₁′, —CONHCH₂CH₂—N(B) (C) (III)

in which:

B represents —CH₂CH₂OX′,

C represents —(CH₂)_{z}—Y′, with z=1 or 2,

X′ represents the group —CH₂CH₂—COOH or a hydrogen atom,

Y′ represents —COOH or the group —CH₂—CHOH—SO₃H,

R_{1′} represents an alkyl group of an acid R_{g}—COOH present in copra oil or in hydrolyzed linseed oil, an alkyl group, in particular a C₁₇ alkyl group and its iso form, an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, there may be mentioned the cocoamphodiacetate marketed under the trade name MIRANOL® C2M Concentrate by the company RHODIA.

Among the amphoteric surfactants, (C₈-C₂₀)alkylbetaines, (C₈-C₂₀) alkylamido (C₆-C₈) alkylbetaines, alkylamphodiacetates and mixtures thereof are preferably used.

The nonionic surfactants which are also suitable in the invention are compounds which are well known per se (see in particular in this regard “Handbook of Surfactants” by M. R. PORTER, published by Blackie & Son (Glasgow and London), 1991, pp. 116-178). Thus, they may be chosen in particular from alcohols, alpha-diols, (C₁-C₂₀) alkylphenols or polyethoxylated, polypropoxylated or polyglycerolated fatty acids, having a fatty chain comprising, for example, from 8 to 18 carbon atoms, it being possible for the number of ethylene oxide or propylene oxide groups to range in particular from 2 to 50 and it being possible for the number of glycerol groups to range in particular from 2 to 30. There may also be mentioned copolymers of ethylene and propylene oxides, condensates of ethylene and propylene oxides with fatty alcohols; polyethoxylated fatty amides preferably having from 2 to 30 mol of ethylene oxide, polyglycerolated fatty amides comprising on average 1 to 5 glycerol groups and in particular 1.5 to 4; polyethoxylated fatty amines preferably having 2 to 30 mol of ethylene oxide; ethoxylated fatty acid esters of sorbitan having from 2 to 30 mol of ethylene oxide; fatty acid esters of sucrose, fatty acid esters of polyethylene glycol, (C₆-C₂₄) alkyl polyglycosides, N-(C₆-C₂₄)alkylglucamine derivatives, amine oxides such as (C₁₀-C₁₄)alkylamine oxides or N-(C₁₀-C₁₄) acylaminopropylmorpholine oxides; and mixtures thereof.

Among the nonionic surfactants cited above, (C₆-C₂₄)alkyl polyglycosides are preferably used.

The composition according to the invention may also comprise one or more cationic surfactants well known per se, such as optionally polyoxyalkylenated primary, secondary or tertiary fatty amine salts; quaternary ammonium salts such as tetraalkylammonium, alkylamidoalkyltrialkylammmonium, trialkylbenzylammonium, trialkylhydroxyalkylammonium or alkylpyridinium chlorides or bromides; imidazoline derivatives; or amine oxides having a cationic character.

The surfactants, as described above, are used in particular in a total quantity of between 4 and 30% by weight, preferably between 6 and 25% by weight, and more preferably between 8 and 22% by weight relative to the total weight of the composition.

The cosmetically acceptable medium may consist of water or a mixture of water and a cosmetically acceptable solvent such as a C₁-C₄ lower alcohol, for example ethanol, isopropanol, tert-butanol, n-butanol; alkylene glycols such as propylene glycol, glycol ethers; C₅-C₁₀ alkanes; acetone, methyl ethyl ketone; C₁-C₄ alkyl acetates such as methyl acetate, ethyl acetate, butyl acetate; dimethoxyethane, diethoxyethane; and mixtures thereof.

The pH of the compositions of the invention is between 3 and 8, preferably between 4 and 7.

The compositions according to the invention may also contain conventional additives well known in the art, such as cationic, nonionic or amphoteric polymers, anionic polymers other than those of the invention, modified or unmodified, volatile or nonvolatile silicones, associative or nonassociative, anionic, amphoteric, zwitterionic, nonionic or cationic, natural or synthetic polymeric thickeners, nonpolymeric thickeners such as acids or electrolytes, opacifiers, perfumes, mineral, vegetable and/or synthetic oils, esters of fatty acids or of polyethylene glycols, colorants, organic or inorganic particles, preservatives, pH-stabilizing agents.

Persons skilled in the art will be careful to choose the optional additives and their quantity so that they do not adversely affect the properties of the compositions of the present invention.

These additives are present in the composition according to the invention in a quantity ranging from 0 to 20% by weight relative to the total weight of the composition.

A preferred class of additives consists of hair-styling polymers and in particular polymers with a polysiloxane backbone which are grafted with hydrocarbon units, and preferably acrylic units.

Another class consists of cationic polymers with vinyllactam units.

The cosmetic compositions according to the invention may be provided in the form of fluid or thickened liquids, of gels, creams, mousses, simple emulsions or multiple emulsions.

They may be used, for example, as shampoos, rinse-out treatments, deep treatment masks, shower gels, lotions or creams for treating the scalp.

The present invention also relates to a method for the cosmetic treatment of keratinous materials which consists in applying to the keratinous materials an effective quantity of a cosmetic composition as described above, and in optionally rinsing after an optional leave-in time.

According to a preferred embodiment of the invention, the composition may be used as a shampoo.

The following examples illustrate the present invention and should not be considered in any manner as limiting the invention.

### EXAMPLES

Two shampoo compositions A and B according to the invention were prepared from the following ingredients:

| Composition | A | B |
|---|---|---|
| Sodium lauryl ether sulfate (C12/C14 at | 10.5 g | 10.5 g |
| 70/30) containing 2.2 mol of ethylene oxide | A.S. | A.S. |
| Amphoteric surfactant derived from | 1.2 g | 1.2 g |
| imidazoline, sold under the name Miranol ® C2M | A.S. | A.S. |
| by the company Rhodia (40% A.S.) | | |
| (C₈/C₁₆) Alkyl polyglycoside (1.4) as a 53% | 8.0 g | 8.0 g |
| aqueous solution sold under the name | A.S. | A.S. |
| PLANTACARE ® 818 UP by the company COGNIS | | |
| Oxyethylenated lauryl alcohol (2.5 mol of | 0.75 g | 0.75 g |
| ethylene oxide) | | |
| Vinylpyrrolidone/methylvinylimidazolium | 1.3 g | 1.3 g |
| chloride (5/95) copolymer as a 40% aqueous | | |
| solution | | |
| Methacrylic acid/acrylic acid/ethyl | 0.2 g | 0.2 g |
| acrylate/methyl methacrylate copolymer as a | A.S. | A.S. |
| 25% aqueous dispersion marketed under the | | |
| name Amerhold ® DR 25 by the company Amerchol | | |
| Crosslinked polyacrylic acid | 0.2 g | 0.2 g |
| Glycol distearate | 3.0 g | — |
| Distearyl ether | — | 1.5 g |
| Mixture of fatty alcohols containing 76% | — | 1.5 g |
| behenyl alcohol marketed under the name | | |
| Nafol ® 1822C by the company Condéa | | |
| Citric acid qs | pH 5 | pH 5 |
| Sodium chloride | 1.5 g | 1.5 g |
| Perfume, preservative | qs | qs |
| Demineralized water qs | 100.0 g | 100.0 g |

A.S.: active substance

Composition A is a composition according to the invention while composition B is a prior art composition.

Both compositions are applied to the hair and the hair is rinsed.

A better volumizing of the hair and imparting of lightness are observed on wet hair with the composition A according to the invention.

1. A cosmetic composition, characterized in that it comprises, in a cosmetically acceptable medium, at least one surfactant, at least one anionic latex, at least one ether having two fatty chains and a mixture of long chain linear and saturated fatty alcohols containing at least 50% by weight of an alcohol which contains 22 carbon atoms.

2. The cosmetic composition as claimed in claim 1, characterized in that the ether having two fatty chains is chosen from ethers having two fatty chains, which are solid at a temperature of less than or equal to about 30° C., and which correspond to the following formula:
R—O—R (I)
in which:
R and R′, which are identical or different, denote a linear or branched, saturated or unsaturated hydrocarbon group containing from 12 to 30 carbon atoms, and preferably from 14 to 24 carbon atoms, R and R′ being chosen such that the compound of formula (I) is solid at a temperature of less than or equal to about 30° C.

3. The cosmetic composition as claimed in claim 1 or 2, characterized in that the ether having two fatty chains is a distearyl ether.

4. The cosmetic composition as claimed in any one of the preceding claims, characterized in that the mixture of fatty alcohols contains at least 50% by weight of an alcohol which contains 22 carbon atoms, relative to the total weight of the mixture.

5. The cosmetic composition as claimed in any one of the preceding claims, characterized in that the anionic latex results from the polymerization of one or more of the following ionogenic monomers: acrylic, methacrylic, maleic, crotonic, itaconic, para-styrenesulfonic, vinylsulfonic, 2-methacryloyloxyethylsulfonic and 2-acrylamido-2-methylpropylsulfonic acids, alone or as a mixture with various monomers such as styrene, butadiene, ethylene, propylene, isoprene, isobutene, vinyltoluene, vinyl propionate, vinyl alcohol, acrylonitrile, chloroprene, vinyl chloride, vinylidene chloride, vinyl acetate, urethanes, vinyl ether, vinylpyrrolidone, vinylimidazole, esters of acrylic, methacrylic, vinylacetic, maleic, crotonic or itaconic acids.

6. The cosmetic composition as claimed in any one of the preceding claims, characterized in that the anionic latex is an aqueous dispersion comprising an acrylic copolymer consisting of (a) about 35 to 74% by weight of an alkyl acrylate, (b) about 25 to 65% by weight of alkyl methacrylate and (c) about 1 to 15% by weight of one or more ethylenic carboxylic acids or their salts, having from 3 to 5 carbon atoms, alkyl groups containing from 1 to 5 carbon atoms, these percentages being expressed by weight relative to the total weight of the copolymer.

7. The cosmetic composition as claimed in claim 6, characterized in that said copolymer comprises from 50 to 60% by weight of ethyl acrylate, from 30 to 40% by weight of methyl methacrylate, from 2 to 10% by weight of acrylic cid, from 2 to 10% by weight of methacrylic acid, the total concentration of acrylic and methacrylic acids not exceeding 15% of the total weight of the copolymer.

8. The cosmetic composition as claimed in any one of the preceding claims, characterized in that the surfactants are chosen from anionic, amphoteric, nonionic and cationic surfactants, and mixtures thereof.

9. The cosmetic composition as claimed in any one of the preceding claims, characterized in that the ether having two fatty chains is used in a quantity of 0.5% by weight to 15% by weight relative to the total weight of the cosmetic composition.

10. The cosmetic composition as claimed in any one of the preceding claims, characterized in that the mixture of long chain linear and saturated fatty alcohols containing at least 50% by weight of an alcohol which contains 22 atoms is used in a quantity of 0.5% by weight to 15% by weight relative to the total weight of the cosmetic composition.

11. The cosmetic composition as claimed in any one of the preceding claims, characterized in that the anionic latex is used in a quantity of 0.05 to 20% by weight of active substances relative to the total weight of the cosmetic composition.

12. The cosmetic composition as claimed in any one of the preceding claims, characterized in that the surfactants are used in a total quantity of 4% by weight to 30% by weight relative to the total weight of the cosmetic composition..

13. The cosmetic composition as claimed in any one of the preceding claims, characterized in that it contains, in addition, a hair-styling polymer.

14. The cosmetic composition as claimed in any one of the preceding claims, characterized in that it contains, in addition, a cationic polymer.

15. A method for the cosmetic treatment of keratinous materials, characterized in that an effective quantity of a cosmetic composition as claimed in any one of the preceding claims is applied to the keratinous materials, and in that rinsing is optionally carried out after an optional leave-in time.

16. The use of a cosmetic composition as claimed in any one of claims 1 to 14, as a shampoo.
